(19) United States
(12) Patent Application Publication
(10) Pub. No.: US 2024/0415861 A1
(43) Pub. Date: Dec. 19, 2024

(54) Preventive Prescription Medication Combination Pack

(71) Applicant: Joseph Michael DeBons, Horseshoe bay, TX (US)

(72) Inventor: Joseph Michael DeBons, Horseshoe bay, TX (US)

(21) Appl. No.: 18/648,514

(22) Filed: Apr. 29, 2024

Related U.S. Application Data
(60) Provisional application No. 63/508,215, filed on Jun. 14, 2023.

(51) Int. Cl. A61K 31/7052; A61K 31/138; A61K 31/192; A61K 31/351; A61K 31/4178; A61K 31/4196; A61K 31/43; A61K 31/433; A61K 31/451; A61K 31/496; A61K 31/5383; A61K 31/5415; A61K 31/573; A61K 31/635; A61K 31/7036

(52) U.S. Cl. CPC A61K 31/7052; A61K 31/138; A61K 31/192; A61K 31/351; A61K 31/4178; A61K 31/4196; A61K 31/43; A61K 31/433; A61K 31/451; A61K 31/496; A61K 31/5383; A61K 31/5415; A61K 31/573; A61K 31/635; A61K 31/7036

(57) ABSTRACT

Preventive prescription medication combination packs offer a proactive approach to healthcare, integrating multiple medications into a single, convenient package and its given in the start of a disease, combining medications enhances adherence by simplifying complex medication regimens, reducing pill burden, and streamlining dosing schedules. This approach is particularly advantageous for preventive care, ensuring consistent medication intake and maximizing therapeutic outcomes because its used for many diseases. Preventive combination packs improve patient convenience, reducing the need for multiple pharmacy visits and minimizing the risk of medication errors. Additionally, they facilitate better communication between patients and healthcare providers, fostering a collaborative approach to treatment.

### FIELD OF THE INVENTION

The present invention relates generally to the field of pharmacy. Specifically, the present invention relates to the provision of qualified patients a preventive medicine pack that has the ability to treat more than 1 medical condition in advance of illness or need of that medication.

### BACKGROUND OF THE INVENTION

Preventive Prescription Medication Combination Pack was first designed and utilized for a patient and elderly relative of Joseph Debons, PharmD. The patient had suffered a variety of illnesses over the past year and needed the prescriptions in advance of holiday travel. Previously, the patient would have had to be reactive to onset of illness or medical condition. Then the patient would have been forced to seek treatment in a foreign location and then obtain the medications from a different pharmacy/pharmacist. The delays in finding and obtaining treatment are both cumbersome and the lack of prompt treatment was just not available or convenient. The pack was so successful that Dr. DeBons then designed a Preventive Prescription Medication Combination Pack for both himself and his wife to take upon traveling. The concept has grown and now a customizable preventive prescription medication pack is now offered by Dr. DeBons.

Previously, medications were a reactive concept upon onset of illness or medical condition. Now a host (more than 1) of medications are available in advance of illness or medical condition. While some preventive prescriptions are in place, they were not offered previously in combination pack to treat a multitude of conditions. Wellness and prompt treatment is provably better with the preventive care package. Therefore, what is needed are techniques that overcome the above mentioned disadvantages.

### SUMMARY OF THE DESCRIPTION

The preventive prescription medication combination pack is able to treat a variety of medical illnesses or conditions in advance of onset of symptoms of those illnesses or conditions, The prescription combination pack is well designed for the following patient groups including (but not limited to) travelling patients, underserved patients, patients who respond well to prompt treatment, patients who prefer discreet telemedicine visits, patients with agoraphobia/iatrophobia, patients who are more comfortable being prepared, patients who cannot leave work or home easily, and patients with transportation issues.

### BRIEF DESCRIPTION OF THE DRAWINGS

FIG. 1 illustrates diagram of an embodiment of a base preventive care pack that could be prescribed by a licensed provider as a preventive prescription medication pack, although every preventive care pack is different and includes medication that a provider sees as fit for that patient.

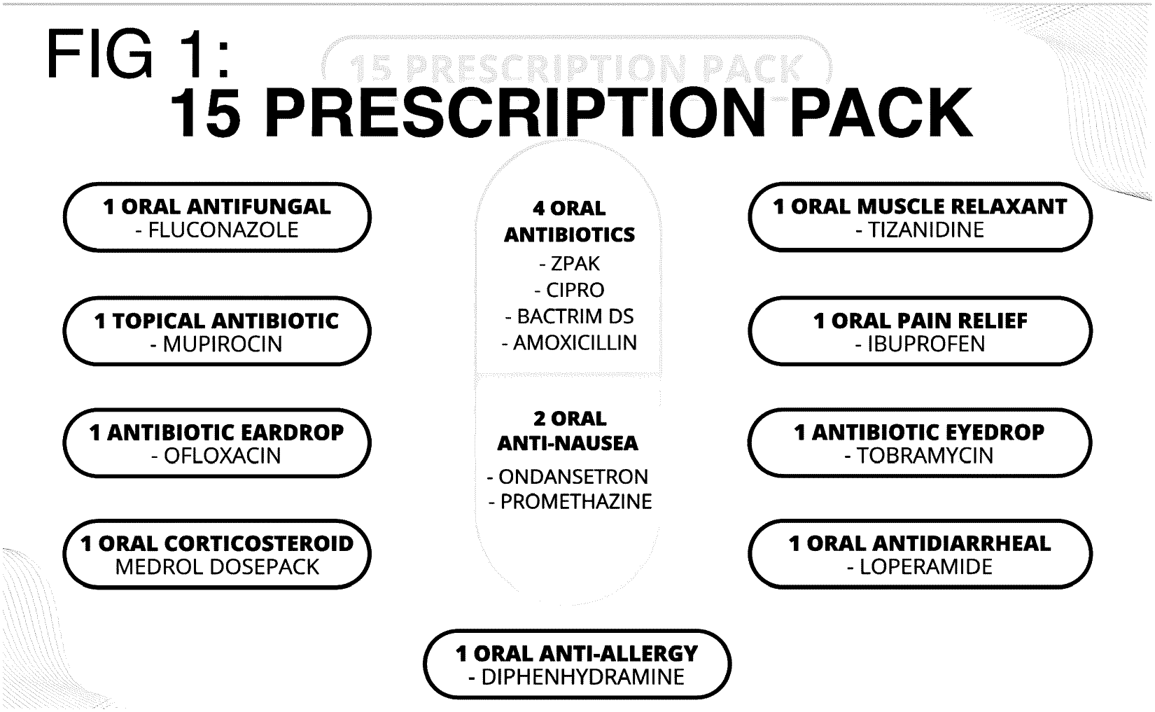

FIG. 2 illustrates diagram of an embodiment of addition add on prescriptions that a provider might prescribe in a customized prescription pack. This is a sample image and more prescriptions could be added but rather this image serves to show some possibilities of add-on prescription therapy.

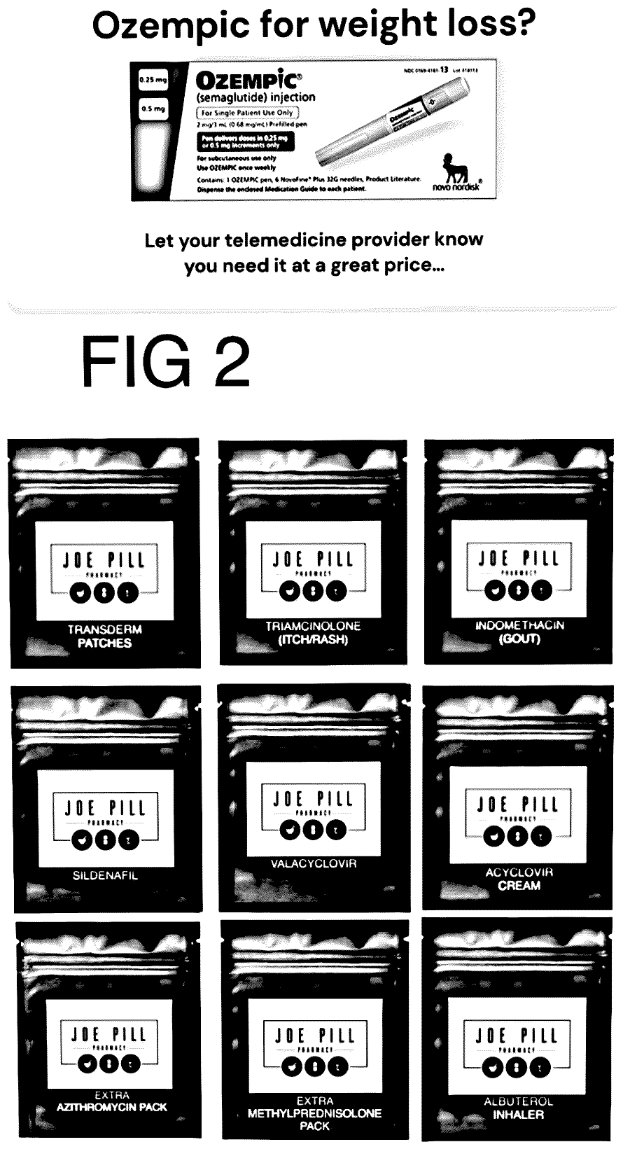

### DETAIL DESCRIPTIONS OF THE INVENTION

All illustrations of the drawings are for the purpose of describing selected versions of the present invention and are not intended to limit the scope of the present invention.

Various embodiments and aspects of the inventions will be described with reference to details discussed below. The following medication descriptions of the invention are not to be construed as limiting the invention, Numerous specific details unique to teach patient are required to provide a thorough understanding of various embodiments of the present invention. However, in certain instances, well-known or conventional details are not described in order to provide a concise discussion of embodiments of the present inventions.

Reference in the specification to “one embodiment” or “an embodiment” or “another embodiment” means that a particular feature, structure, or characteristic described in conjunction with the embodiment can be included in at least one embodiment of the invention. The appearances of the phrase â€œin one embodimentâ€ in various places in the specification do not necessarily all refer to the same embodiment. The following medical treatments can be included in the preventive prescription pack as deemed beneficial by the medical provider.

The pack is unique in that the patient treatment is deemed necessary and a host (more than 1) prescription is deemed necessary and included in the preventive care pack. The medications that are considered for a preventive prescription care pack

(embodiment (include (but are not limited to) the following:
1) Antibiotics
2) Breathing Medications
3) Bone disease Prescriptions
4) Prescriptions for Teeth/Oral Care
5) Treatments for Colonoscopy
6) Diabetic Medication
7) Heart Disease Prescriptions
8) Cholesterol Lowering Medications
9) High Blood Pressure Medications
10) Travel Medications (i.e. malaria)
11) Antivirals
12) Migraine Prevention
13) Obesity Medications
14) Smoking Cessation Medications
15) Muscle Relaxants
16) G1 treatments including nausea, vomiting, heartburn, diarrhea, indigestion, antacids
17) Steroid Packs i.e. Medrol Dose Pack or Prednisone Pack or Other
18) Antifungals
19) Pain relievers
20) Allergy medications
21) Birth Control
22) Acne Prevention
23) Wrinkle Prevention
24) Erectile Dysfunction
25) Skin Care
26) Gout Treatment
27) Anti-inflammatory medications
28) Cold Sore Prevention/Treatment
29) Sea Sickness Prevention

The prescriptions may be oral, optic, ophthalmic, mucosal, transdermal, or topical as seen as medically necessary per prescriber.

Although the invention has been explained in relation to its preferred embodiment, it is to be understood that many other possible modifications and variations can be made without departing from the spirit and scope of the invention.

1. Preventive prescription medication pack that has the ability to treat more than one medical condition in advance of illness or need of that medication. Prescription pack is provided pursuant to telemedicine visit, eliminating the needs and risks of waiting rooms at both doctor's offices and pharmacies. Such preventive care allows for prompt treatment to improve patient outcome(s) quickly. Other benefits include, but are not limited to: traveling patients, underserved patients, patients who respond to prompt treatment, patients who prefer discreet medical care, patients who cannot leave work or home easily, and patients with limited transportation.
